(19) United States
(12) Patent Application Publication
(10) Pub. No.: US 2019/0185814 A1
(43) Pub. Date: Jun. 20, 2019

(54) DEVICE AND METHODS FOR TISSUE PROCESSING

(71) Applicant: LifeCell Corporation, Madison, NJ (US)

(72) Inventor: Nathaniel Bachrach, Clifton, NJ (US)

(21) Appl. No.: 16/218,992

(22) Filed: Dec. 13, 2018

Related U.S. Application Data
(60) Provisional application No. 62/598,754, filed on Dec. 14, 2017.

(51) Int. Cl. C12N 5/077; A61M 5/20; A61M 1/00; A61L 27/36

(52) U.S. Cl. CPC C12N 5/0653; A61M 5/20; A61L 27/3604; A61M 1/0094; A61M 1/0056

(57) ABSTRACT

The present disclosure provides devices and methods for processing and harvesting adipose tissue. The disclosed devices, systems, and methods offer allow for filtering and harvesting small adipose particles. The devices, systems, and methods include a device comprising an interior tissue harvesting volume and an exterior waste collection volume separated by a filter. Fluids and small molecular elements that pass through the filter during tissue processing are removed, while adipose tissues of interest may remain within the filter to be collected and reinjected.

This application claims priority under 35 USC § 119 to U.S. Provisional Application No. 62/598,754, which was filed on Dec. 14, 2017 and is herein incorporated by referenced in its entirety.

The present disclosure relates to devices, systems, and methods for processing tissue, and, more particularly, to processing and harvesting adipose tissue.

Autologous fat transfer is a procedure that involves harvesting adipose tissue from one region of a patient's body, typically by aspiration, and implanting the harvested adipose tissue in another region of the patient's body. Autologous fat transfer has numerous clinical applications such as facial contouring, breast reconstruction and/or augmentation, buttock augmentation, and other aesthetic or reconstructive procedures. In addition, autologous fat grafting has been found to have relatively low donor-site morbidity, as compared with other surgical options.

Autologous fat facial contouring is a popular procedure that involves injecting small volumes of adipose tissue in a patient's face to correct, enhance, or reconstruct facial features. Facial contouring may include, for example, lip augmentation procedures or the treatment of facial rhytids, such as nasolabial folds, mesolabial folds, oral commissures, periorbital lines, and glabellar lines. To reduce patient discomfort and to minimize scarring, small-gauge needles are often used in facial contouring, which require smaller particle sizes and low-viscosity materials for successful operation.

Existing devices for processing adipose tissue, while effective for large-volume fat injection applications, may produce filtrate that is ill-suited for the small-gauge needles commonly used in autologous fat facial contouring procedures. For example, existing devices that process lipoaspirate collected from the donor site of a patient may be used to filter saline, tumescent material, blood, and other extraneous materials from the remaining material, mostly comprising adipose tissue particles (i.e. adipocytes). In this process, small adipose tissue particles that are suitable for small-volume injections, may be inadvertently removed. As such, a filtration device that may harvest small adipose tissue particles from lipoaspirate would be useful in procedures where small adipose tissue particle injection is well suited, such as autologous fat facial contouring.

Accordingly, the present disclosure provides devices and methods for cleaning and harvesting adipose tissue. Particularly, the disclosed devices, systems, and methods offer a means for processing and harvesting small tissue volumes that can be readily reinjected.

### SUMMARY

The present disclosure provides devices, systems, and methods for improved tissue processing. The devices, systems, and methods may be used to filter adipose material collected from a donor site prior to injection elsewhere in the patient's body, for example, in small-volume injection areas, such as the face and neck.

A device for processing adipose tissue is provided according to various embodiments described herein. The device can include a fluid pathway comprising a first end, a second end, and a lumen extending between the first end and the second end for passage of fluid therebetween. The device also includes a first filter disposed within the fluid pathway between the first end and the second end, the filter being configured to collect adipose tissue particles and selectively filter waste material from the fluid. The device also comprises a waste collection volume surrounding the filter and includes a waste port for engagement with a waste removal device. A tissue retrieval port in fluid communication with the filter proximate the second end of the fluid pathway is provided.

A system for processing adipose tissue is provided according to various embodiments described herein. The system includes a tissue container and processing device with an exterior wall surrounding an interior volume for holding adipose tissue, a structure for processing the adipose tissue, and at least one transfer port. The system further comprises a device for processing filtrate from the tissue container. The device for processing filtrate from the tissue container comprises a fluid pathway comprising a first end, a second end, and a lumen extending between the first end and second end for passage of the fluid therebetween. The device for processing filtrate from the tissue container also includes a first filter disposed within the fluid pathway between the first end and the second end, the filter being configured to collect adipose tissue particles and selectively filter waste material from the fluid. Additionally, the device for processing filtrate from the tissue container includes a waste collection volume surrounding the filter and including a waste port for engagement with a waste removal device, and a tissue retrieval port in fluid communication with the filter proximate the second end of the fluid pathway. The system for processing adipose tissue can also include a container for collecting adipose tissue, which can connect to the system such that it is in fluid communication with the tissue retrieval port.

A method of processing adipose tissue is provided according to various embodiments described herein. The method comprises selecting a device comprising a fluid pathway including a first end, a second end, and a lumen extending between the first end and second end for passage of a fluid therebetween. The device also includes a first filter disposed within the fluid pathway between the first end and the second end, the filter being configured to collect adipose tissue particles and selectively filter waste material from the tissue. The device further includes a waste collection volume surrounding the filter and including a waste port for engagement with a waste removal device. The device further comprises a tissue retrieval port in fluid communication with the filter proximate the second end of the fluid pathway. The method of processing adipose tissue further comprises advancing the fluid through the fluid pathway, evacuating waste material through the waste port into the waste removal device, and removing collected adipose tissue particles into a container for collection of adipose tissue.

### BRIEF DESCRIPTION OF THE DRAWINGS

FIG. 1 illustrates a partial cross-sectional view of a device for processing filtrate, according to various embodiments of the present disclosure.

FIG. 2 illustrates a partial cross-sectional view of two devices for processing filtrate configured in series, according to various embodiments of the present disclosure.

FIG. 3A illustrates a magnified, cross-sectional view of the boundary between a filter and waste collection volume of the device from FIG. 2, according to various embodiments of the present disclosure.

FIG. 3B illustrates a magnified, cross-sectional view of the boundary between a filter and waste collection volume of the device from FIG. 2, according to various embodiments of the present disclosure.

FIG. 4 illustrates a perspective view of a system for processing adipose tissue including a device for processing filtrate connected to a tissue container and processing device.

### DESCRIPTION OF CERTAIN EXEMPLARY EMBODIMENTS

Reference will now be made in detail to certain exemplary embodiments according to the present disclosure, certain examples of which are illustrated in the accompanying drawings. Wherever possible, the same reference numbers will be used throughout the drawings to refer to the same or like parts.

In this application, the use of the singular includes the plural unless specifically stated otherwise. In this application, the use of “or” means “and/or” unless stated otherwise. Furthermore, the use of the term “including,” as well as other forms such as “included” and “includes,” is not limiting.

The section headings used herein are for organizational purposes only and are not to be construed as limiting the subject matter described. All documents, or portions of documents, cited in this application including but not limited to patents, patent applications, articles, books, and treatises are hereby expressly incorporated by reference in their entirety for any purpose.

The use of the word “syringe” is not limited to any industry standard and includes any of a variety of receptacles provided in different shapes and sizes. Any range described herein will be understood to include the endpoints and all values between the endpoints.

As used herein, “tissue processing” can refer to a number of steps or treatments intended to clean or process tissue. Such steps can include washing, removal of collagen strands, mechanical agitation or separation, or removal or filtration of waste and wash from harvested tissue.

As used herein, “adipose tissue” refers to adipose tissue obtained by any means including, for example, liposuction and/or tumescent liposuction. The adipose tissue can be autologous tissue, allogeneic tissue, or xenogenic tissue (e.g., bovine or porcine tissue). Additionally, adipose tissue can refer to particles with multiple adipocyte cells included therein.

As used herein, “small volume” generally refers to volumes of the order of 300 ml or less. Although, it can be appreciated that as autologous fat transfer procedures advance and evolve, the volumes used for facial and other small-volume injection sites may change.

Various human and animal tissues can be used to produce products for treating patients. For example, various tissue products have been produced for regeneration, repair, augmentation, reinforcement, and/or treatment of human tissues that have been damaged or lost due to various diseases and/or structural damage (e.g., from trauma, surgery, atrophy, and/or long-term wear and degeneration).

Fat grafting, including autologous fat grafting, involves collecting adipose tissue from a donor site and injecting the collected tissue into a host site. Fat grafting can be useful for a variety of clinical applications, including large-scale facial fillers, breast augmentation, buttock augmentation/sculpting, augmentation of other tissue sites, correction of lumpectomy defects, cranial-facial defect correction, and correction of lipoplasty defects (divots).

Autologous fat grafting can also be used in small-volume applications such as lip augmentation, and procedures addressing facial scars and rhytids (i.e. nasolabial folds, mesolabial folds, oral commissures, periorbital lines, and glabellar lines). To reduce patient discomfort and to minimize scarring, small-gauge needles are often used in facial autologous fat transfer, which require low-viscosity injectate comprising small, particulate tissue for successful administration.

Autologous fat grafting procedures start with the collection of adipose tissue from a donor site. To effectively remove adipose tissue from the donor site, collection techniques may include water-assisted liposuction and tumescent liposuction, and may involve injecting a donor site with a liquid used to loosen adipose tissue so that it can be aspirated into a cannula connected to suction and collection devices. The collected material, thus, may include adipose tissue, whole adipocytes, lysed adipocytes, water, saline, tumescence, blood, and blood vessel tissues, among other materials. The aspirated material may be processed such that the remaining material predominantly contains washed adipose tissue, and results in a suitable injectate.

Devices, systems, and methods disclosed herein allow for the filtration and harvest of adipose tissue (i.e. lipoaspirate) collected from a donor site. The devices, systems, and methods of the present disclosure are well suited for harvesting small adipose particles that are appropriate for use in small-volume injection sites of the body, such as the rhytids or deformities of the face and neck.

In various embodiments, the devices, systems, and methods of the present disclosure may be combined with a variety of existing adipose tissue processing devices, such as the REVOLVE™ fat grafting system from LIFECELL™ CORPORTATION (MADISON, N.J.). While existing devices may be effective for large-volume fat injection applications, it may be desirable to process the filtrate from such devices to generate optimal injectate for small-volume applications. Additionally, in some instances, autologous fat transfer patients have minimal amount of adipose tissue available for harvesting. As such, a system that provides a second filtration of the lipoaspirate can increase the total volume of adipose tissue available for injection.

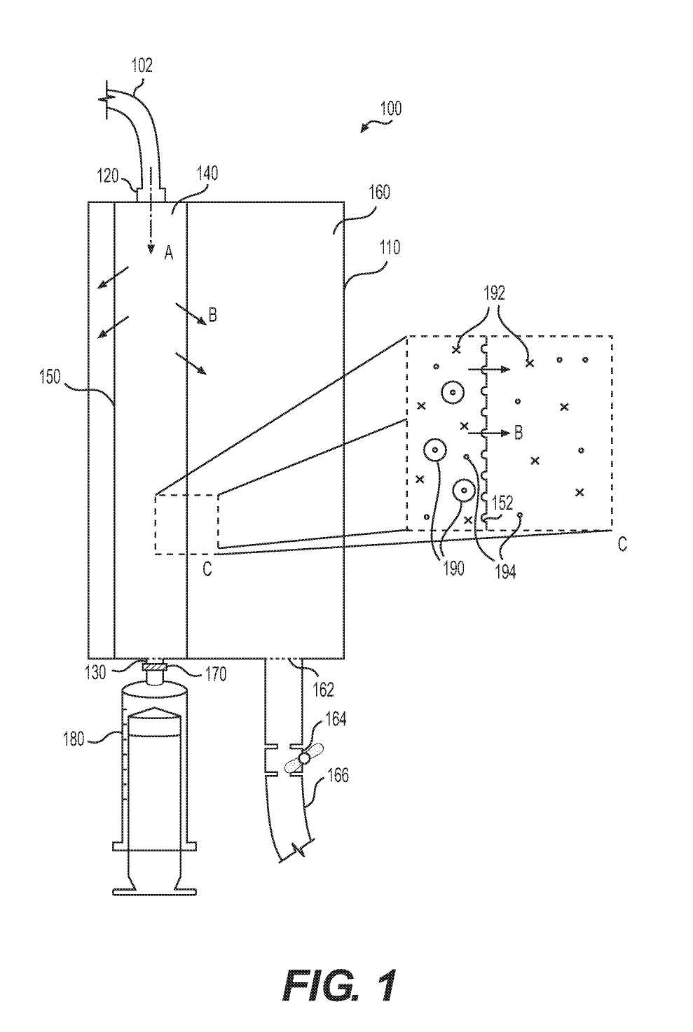

FIG. 1 illustrates a cross-sectional view of a device for processing filtrate 100, in accordance with various embodiments of the present disclosure. In some embodiments, device for processing filtrate 100 (hereinafter ‘device 100’) may be used in processing material with small, particulate adipose tissue to harvest adipose tissue 190 and remove small undesired components 192, 194 and fluids. Device 100 may include fluid pathway (A) comprising first end 120, second end 130, and lumen 140 extending between first end 120 and second end 130 for passage of fluid therebetween. Device 100 may also include first filter 150 (hereinafter “filter 150”) disposed within fluid pathway (A) between first end 120 and second end 130. In various embodiments, filter 150 is configured to collect adipose tissue 190 and selectively filter fluids and waste materials 192, 194.

Additionally, device 100 may include waste collection volume 160 surrounding filter 150 and include waste port 162 for engagement with waste removal device 166. In various embodiments, device 100 comprises tissue retrieval port 170 that is in fluid communication with filter 150 proximate second end 130 of fluid pathway (A). Tissue retrieval port 170 may also provide a means to connect adipose collection container 180 to second end 130 of fluid pathway A.

In various embodiments, device 100 may be provided in a variety of sizes and configurations. In certain embodiments, device 100 may be sized to accommodate a range of tissue volumes. For example, device 100 may be configured to accommodate tissues volumes of 0.5, 1, 2, 3, 4, 5, 10, 20, 30, 40, 50, 60, 70, 80, 90, 100, 120, 140, 160, 180, 200, 220, 240, 260, 280, 300, 350, 400, 450, 500, or 1000 ml, or ranges in between. These tissue volume values may be used to define a single tissue volume, such as 80 ml, or may be used to define range of tissue volumes, such as from about 100-120 ml. The tissue volume of device 100 may be configured to accommodate a desired volume and rate of tissue input into device 100.

In various embodiments, device 100 may be produced from in a variety of materials suitable for tissue processing. For example, device 100 may be manufactured from materials that will enable the device to pass regulatory testing standards, such as ISO 10993-1. Such materials may be sufficiently biocompatible and inert as to not elicit cytotoxic responses during clinical use. Examples of materials potentially suitable for device 100 may include plastics, such as polymers (e.g. polyethylene terephthalate (PET), high density polyethylene (HDPE), polyvinyl chloride (PVC), polypropylene (PP), polyimide (TPI), and acrylonitrile butadiene styrene (ABS)), metals (e.g. stainless steel, titanium alloys, cobalt chromium, nitinol, and copper), or ceramics (e.g. zirconia, or alumina).

The materials of device 100 must be able to withstand stresses of the manufacturing and sterilization processes, and well as stresses endured during clinical use. For example, the materials of device 100 may need to be able to withstand high sterilization temperatures, or negative pressures generated from suction devices used during the procedure.

Additionally, in certain embodiments, device 100 may comprise one or more materials configured to improve user-experience. For example, outer surface 110 of device 100 may comprise a transparent material so that users (e.g. surgeons or other medical professionals) may view tissue or other internal components during use. Furthermore, materials of device 100 may be optimized to minimize cost or to simplify the device manufacturing process.

In various embodiments, lipoaspirate is input into device 100 through inlet tube 102. In some embodiments, device 100 comprises fluid pathway (A) including first end 120 and second end 130. Lumen 140 extends between first end 120 and second end 130 for the passage of fluid. Fluid pathway (A) illustrates the direction of fluid flow entering device 100. Lumen 140 may extend partially or completely along the height of device 100 and may be provided in a variety of configurations. For example, lumen 140 may be provided in a straight configuration oriented vertically, or may be provided in a helical configuration also oriented vertically. However, additional orientations and configurations of lumen 140 are contemplated in the present disclosure.

In various embodiments, inlet tube 102 may comprise a hose, tube, cannula, or other passageway that includes a lumen through which fluid, such as lipoaspirate or filtrate materials from an outside source (e.g. in direct contact with the donor site, or separate collection or filtration device), may travel. In various embodiments, inlet tube 102 may be provided in a variety of materials, for example, plastic, silicone, nylon or rubber (e.g. latex). Plastic tubing may comprise polyvinyl carbonate (PVC), a polyolefin, a polyurethane, polyethylene, polypropylene, or a fluoropolymer (e.g. PTFE, FEP, PFA).

In various embodiments, the shape of lumen 140 may be defined by filter 150. As such, filter 150 may serve as a boundary between lumen 140 and waste collection volume 160. Filter 150 may be configured to allow fluids and small undesired components 192, 194 (e.g., chemicals, blood, non-viable proteins) to pass through while preventing passage of tissue components, such as adipose tissue 190. Section C of FIG. 1 illustrates a magnified view of the boundary between lumen 140 and waste collection volume 160. In various embodiments, as illustrated in section C, adipose tissue 190 (some or all based on size or other factors) may remain within lumen 140 (left) while fluids and small undesired components 192, 194 may pass through pores 152 of filter 150, and enter into waste collection volume 160 (right) along direction B. In various embodiments, the filtration of lipoaspirate within device 100 may be a hybrid of tangential and flatbed filtration.

In various embodiments, filter 150 may be provided in a variety of forms. For example, filter 150 may be formed from a variety of materials comprising one or more pores 152. In some embodiments, filter 150 may be formed from a mesh material such as a porous polymer mesh or metal mesh. In some embodiments, filter 150 may comprise a screen or netting. Filter 150 may be rigid or pliable in various embodiments. Pores 152 of filter 150 may be provided in a variety of sizes suitable for the retention of desired adipose tissue particles. For example, pores 152 may be about 1, 2, 3, 4, 5, 10, 20, 30, 40, 50, 100, 150, 200, 250, 300, 350, 400, or 500 μm in size. The listed sizes may comprise a single pore 152 size, such as 50 μm, or may be used to define a range of pore 152 sizes, such as 100-150 μm.

In some embodiments, filter 150 may be positioned in various locations within device 100. For example, filter 150 may be disposed within fluid pathway (A) between first end 120 and second end 130. Filter 150 may have a substantially uniform or varying cross section along its length. For example, filter 150 may comprise a substantially cylindrical or polyhedron prismatic shape, resulting in a substantially uniform cross section along its length, or may comprise a conical or tetrahedral shape, resulting in a varying cross section along its length. In some embodiments, device 100 may contain more than one filter 150 and fluid pathways (A) to expedite filtration or improve adipose tissue yield rates. The multiple filters 150 may be similarly shaped and uniformly disposed within device 100, or may vary in shape and have a random or patterned disposition within device 100.

In various embodiments, adipose tissue may collect within filter 150 toward second end 130. Adipose collection container 180 is connected to second end 130 of device 100 via adipose retrieval port 170. In various embodiments, adipose retrieval port 170 is configured to enable controlled fluid communication between adipose tissue 190 within filter 150 and adipose collection container 180. Adipose retrieval port 170 may be provided in a variety of configurations. For example, adipose retrieval port 170 may comprise a means for controlling flow, such as a stopcock, Tuohy borst, gate valve, globe valve, ball valve, or needle valve, among others. Additionally, adipose retrieval port 170 may be permanently or removably attached to device 100. In some embodiments, adipose retrieval port 170 may be configured to allow adipose tissue to flow into adipose collection container 180 without causing damage to adipose tissue 190.

Adipose collection container 180 may be configured to efficiently collect adipose tissue from device 100. For example, in some embodiments, adipose collection container 180 may comprise a customized or commercially available syringe. In some embodiments, after adipose tissue 190 harvesting is complete using device 100, adipose retrieval port 170 may be opened, establishing fluid communication between filter 150 and adipose collection container 180. Further, negative pressure may be applied to the syringe body by withdrawing a plunger. Afterwards, adipose tissue 190 within filter 150 proximate second end 130 may flow into the barrel of the syringe, (i.e. adipose collection container 180).

In various embodiments, adipose collection container 180 may be provided to comprise a variety of collection volumes. For example adipocyte collection container 180 may include a collection volume of about 5, 10, 15, 20, 25, 30, 35, 40, 45, 50, 100, 150, 200, 250, 300, 350, 400, 450, or about 500 ml. The collection volume of adipocyte collection container 180 may be configured to contain a volume of adipose tissue appropriate for small-volume fat transfer procedures, such as autologous fat facial transfer.

During use of device 100, waste material and fluids may accumulate within waste collection volume 160. To remove waste material and fluids from device 100, in various embodiments, device 100 includes a device or means for disposing of waste lipoaspirate components or fluids. As illustrated in FIG. 1, waste collection volume 160 includes waste port 162 for engagement with waste removal device 166. Waste collection volume 160 may be provided in a variety of shapes configured to collect waste material in accordance with methods of the present disclosure. In some embodiments, waste collection volume 160 may be defined by outer surface 110 of device 100.

In various embodiments, device 100 is oriented vertically with waste port 162 positioned at the bottom of outer surface 110. In this configuration, device 100 may rely on gravity to transfer lipoaspirate waste material and fluids from waste collection volume 160, through waste port 162, and into waste removal device 166. In some configurations, waste removal device 166 may apply negative pressure to waste collection volume 160 in order to move lipoaspirate waste material and fluids toward waste port 162 and into waste removal device 166. Waste removal device 166 may generate negative pressure through a variety of means, including, peristaltic pump, hydraulic pump, or piston mechanism, among others.

In order to control the flow of waste material within waste collection volume 160, waste port 162 may include a waste port valve 164. Waste port valve 164 may be provided in a variety of configurations, including as a stopcock, Tuohy borst, or other valve type. In various embodiments, waste removal device 166 is removably attached to waste port valve 164. During use of device 100, if waste removal device 166 becomes full and requires emptying, an operator may manipulate waste port valve 164 to stop the flow of material from waste collection volume 160 though waste port valve 164. This would allow a user to remove waste removal device 166 from device 100 and empty its contents. Once emptied, waste removal device 166 may be reattached to waste port valve 164 and, waste port valve 164 may be manipulated to reestablish fluid communication between waste collection volume 160 and waste removal device 166.

In certain embodiments, adipose retrieval port 170 may remain in a closed configuration during lipoaspirate processing. Simultaneously, waste port valve 164 may remain open to allow waste materials to exit device 100. To expedite waste removal, in various embodiments, waste removal device 166 may comprise a negative pressure system to draw waste material from waste collection volume 160 into waste removal device 166. Once waste materials have been removed from device 100, waste port valve 164 may be placed in a closed position, and adipose retrieval port 170 may be placed in an open configuration. With adipose retrieval port 170 in an open position, adipose tissue 190 within filter 140 may move into adipose collection container 180. Additionally, negative pressure may be applied to adipose collection container 180, (i.e. by withdrawing a syringe plunger or activating a negative pressure system) to advance adipose tissue 190 within filter 140 into adipose collection container 180.

To minimize the risk of adipose tissue contamination, adipose collection container 180 may be provided in a sterile state and may be configured for use in clinical procedures, such as autologous fat facial transfer. For example, adipose collection container 180 may be provided as a syringe onto which an injection needle may be attached. Users may proceed to re-inject autologous adipose tissue into the donor site of a patient, such as a rhytid on the face or neck. To perform the procedure, the syringe needle pierces the skin of the patient at an injection site, and a syringe plunger can be depressed into the barrel of the syringe to expel adipose tissue 190 from syringe 180 into the injection site.

In certain applications, it may be desirable to filter adipose tissue into multiple adipose tissue size categories. For example, a user may want adipose tissue in the 100-150 μm particle size range for transfer into deep facial rhytids, or for use in feature enhancement procedures, such as cheek enhancement. Alternatively or additionally, users may desire adipose tissue in the 50-80 μm particle size range for injection into shallow facial rhytids or small scars or deformities. To achieve distinct levels of adipose tissue separation by particle size, two devices of the present disclosure may be connected in series, with filters of each device configured to retain adipose particles of different sizes.

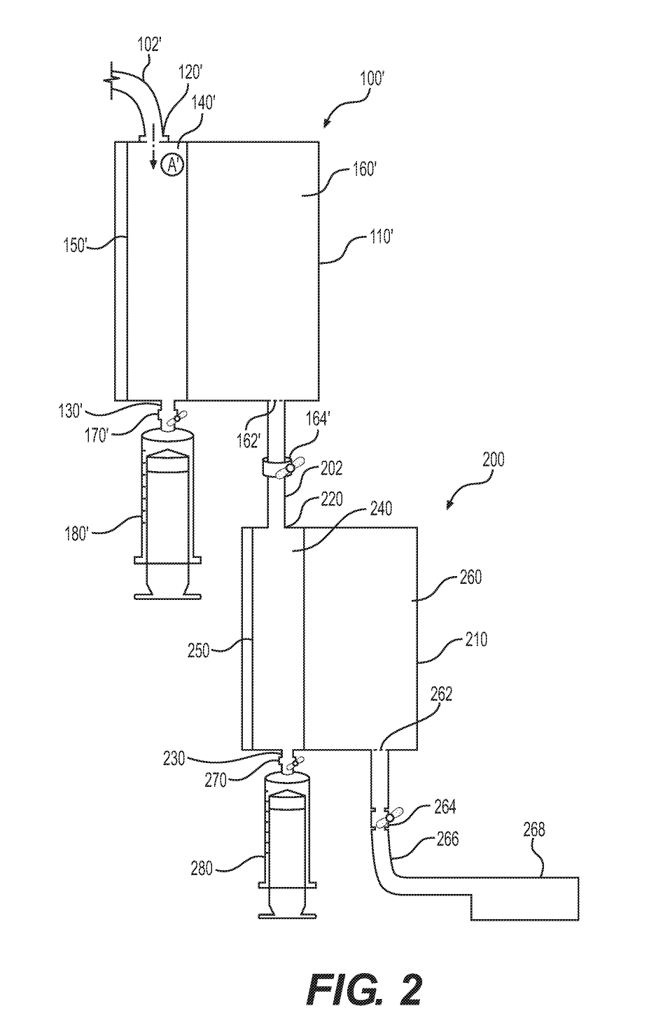

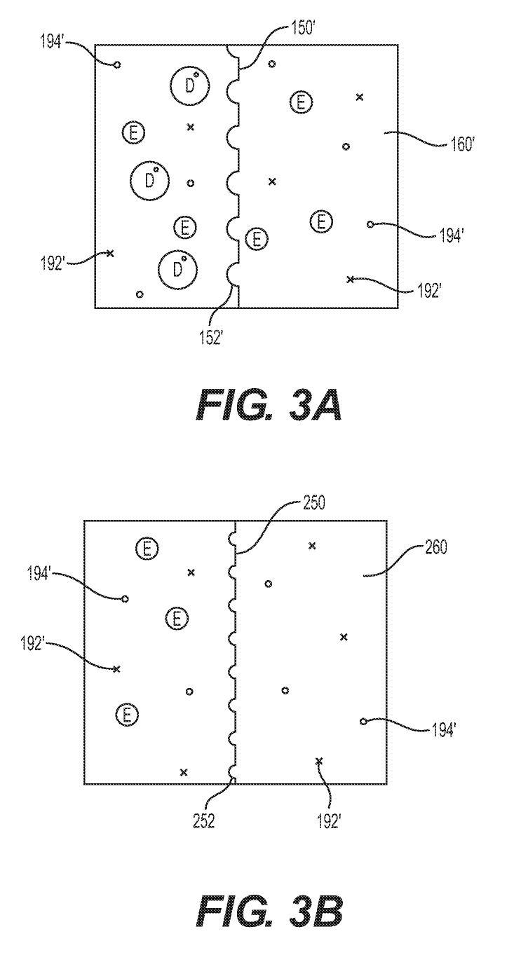

FIG. 2 illustrates a partial cross-sectional view of two devices for processing filtrate configured in series, according to various embodiments of the present disclosure. FIGS. 3A and 3B illustrate magnified, cross-sectional views of boundaries between filters and waste collection volumes of the devices from FIG. 2, according to various embodiments of the present disclosure. In some embodiments, filtrate materials enter device 100′ through inlet tube 102′. Filtrate materials then enter lumen 140′ and contact filter 150′, which may be configured to retain adipose tissue with particles sizes larger than the pore size of filter 150′ (adipose tissue D illustrated in FIG. 3A). Filter 150′ can allow fluids, small molecular elements, and adipose tissue smaller than the pore size of filter 150′ (adipose tissue E illustrated in FIG. 3A) to pass through its pores 152′. In various embodiments, adipose tissue D would remain within filter 150′, and via gravity or a negative pressure system, would migrate toward second end 130′ of device 100′. Adipose tissue may then be processed in preparation for injection into an injection site.

In various embodiments, adipose retrieval port 170′ maybe opened so that adipose collection container 180′ is placed in fluid communication with filter 150′. Additionally, a pressure system may be used to generate negative pressure within adipose collection container 180′ (e.g. the retraction of a syringe plunger) so that adipose particles within filter 150′ may travel into adipose collection container 180′. In various embodiments, adipose retrieval port 170′ can be closed so that adipose collection container 180′ may be removed from device 100′.

In various embodiments, filtrate, which may include lipoaspirate, fluids, small molecular elements and adipose tissue E that flow through the pores 152′ of filter 150′, collect in waste collection volume 160′. Next, in various embodiments, the filtrate described above may flow toward waste port 162′. In an open configuration, waste port valve 164′ allows the contents of waste collection volume 160′ to flow through waste port valve 164′ into connecting tubing 202 and lumen 240 of device 200.

In various embodiments, upon entering lumen 240, lipoaspirate materials contact filter 250, whose pore size may be configured to retain adipose tissue E (illustrated in FIG. 3B) while allowing lipoaspirate fluids and small molecular elements to pass through the pores of filter 250 into waste collection volume 260. In various embodiments, adipose tissue E remains within filter 250, and via gravity or a negative pressure system, migrates toward second end 230 of device 200.

In various embodiments, waste port valve 264 may be manipulated into an open configuration, allowing fluid communication between waste collection volume 260 and waste removal device 266. The materials contained within waste collection volume 260 can then flow through waste port 262 and into waste removal device 266. In various embodiments, collection chamber 268 can be used to contain the waste materials, which may consist primarily of fluids (e.g. saline or water) and small molecular elements.

In various embodiments, waste port valve 264 can be closed, and adipose retrieval port 270 can be opened to place adipose collection container 280 in fluid communication with filter 250. To expedite adipose tissue extraction from filter 250, a negative pressure system may be used to generate negative pressure within adipose collection container 280 (e.g. the retraction of a syringe plunger). In various embodiments, adipose retrieval port 270 may be closed so that adipose collection container 280 may be removed from device 200. Further, a user (e.g. a surgeon) can attach an injection needle to adipose collection container 280 for use in autologous fat facial transfer procedures.

To illustrate the functional and structural differences between devices 100′ and 200 of FIG. 2, a magnified view of the boundaries between filter 150′ and waste collection volume 160′, and between filter 250, and waste collection volume 260 are magnified and illustrated in FIGS. 3A and 3B, respectively.

FIG. 3A illustrates a magnified view of the boundary between the filter 150′ and waste collection volume 160′ of device 100′ from FIG. 2, according to various embodiments of the present disclosure. In FIGS. 3A and 3B, the particles and pores are generally circular, so their characteristic lengths would be represented by their respective diameters. However, other particle and pore shapes are contemplated within the present disclosure and, as such, their characteristic lengths could be some other dimension, such as height or width.

During use of device 100′, as filtrate enters lumen 140′ and contacts filter 150′, certain elements of the filtrate may pass through pores 152′ of filter 150′. As illustrated in FIG. 3A, fluids and small undesired components 192′ and 194′ of the filtrate pass through pores 152′ into waste collection volume 160′. Pores 152′ are sized to retain adipose tissue D, which has a characteristic length (e.g. diameter) larger than the diameter of pores 152′. However, adipose tissue E, which has a diameter smaller than the diameter of pores 152′, may pass through pores 152′ and enter waste collection volume 160′.

In various embodiments, device 100′ retains adipose tissue D within filter 150′ while fluids, small undesired components 192′ and 194′, and adipose tissue E pass to waste collection volume 160′. Device 100′ enables adipose tissue that is larger than pores 152′ to be harvested in filter 150′ and removed from device 100′ for further use.

FIG. 3B illustrates a magnified view of the boundary between filter 250 and waste collection volume 260 of device 200 from FIG. 2, according to various embodiments of the present disclosure. In some embodiments, filtrate from waste collection volume 160′ may travel through waste port 162′ and waste port valve 164′, into connecting tubing 202. Next, the lipoaspirate may pass through first end 220 and enter lumen 240 of device 200.

In various elements, as the filtrate contacts filter 250, certain elements may pass through pores 252 of filter 250. As illustrated in FIG. 3B, fluids and small undesired components 192′ and 194′ of the filtrate pass through pores 252 into waste collection volume 260. Pores 252 are sized to retain adipose tissue E that has a diameter larger than the diameter of pores 252.

Providing multiple filtration and harvesting elements in a single system, as illustrated in FIG. 2, enables precise filtration and harvesting of adipose tissue by particle size. As such, users may provide more customized and advanced injection treatments to patients, particularly in very visible areas where surgical scars may be more noticeable, such as the face and neck.

In various embodiments, multiple devices 100′ and 200 of FIG. 2 may be connected in series or in parallel to enable greater options for harvesting adipose tissue by size. As such, embodiments of the present disclosure are not limited to two devices. It is within the scope of the present disclosure to include 3, 4, 5, 6, 7, 8, 9, 10 or more devices in series or parallel to achieve a more refined adipose particle separation system.

The devices of the present disclosure may be used in conjunction with existing adipose tissue processing devices. For example, devices 100, 100′, and 200 may be connected by some means to alternative filtration systems, such as those with pore sizes larges than pores 152, 152′, and 252.

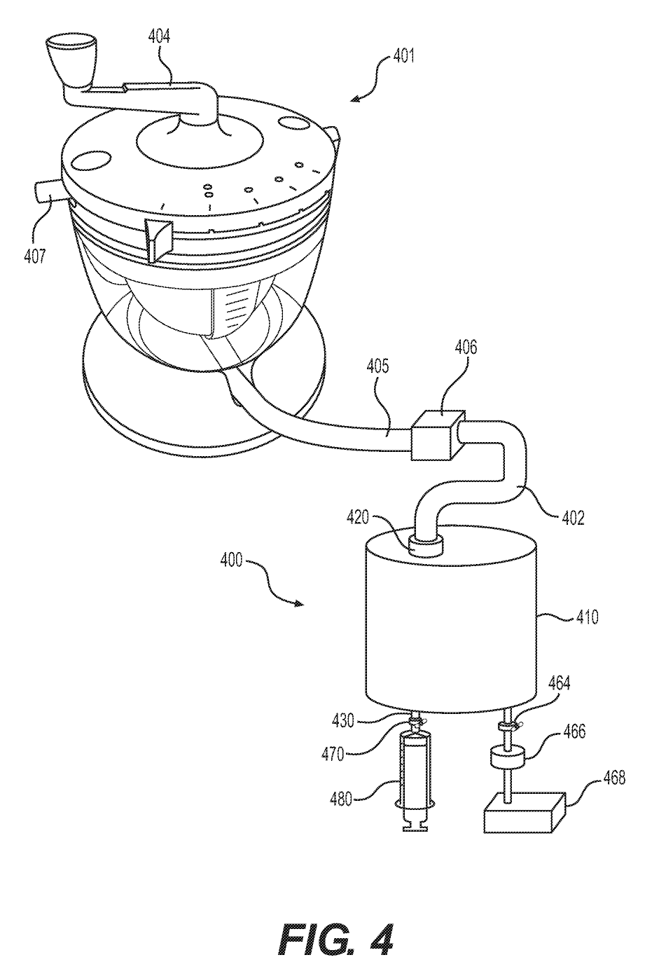

FIG. 4 illustrates a perspective view of a system for processing adipose tissue including a device for processing filtrate connected to a commercially available adipose tissue container and processing device. In various embodiments, adipose tissue container and processing devices such as the REVOLVE™ fat grafting system from LIFECELL™ CORPORTATION (BRANCHBURG, N.J.), can be paired with filtrate processing devices of the present disclosure. In various embodiments, representative adipose container and processing device 401 (hereinafter ‘device 401’) may comprise a means for operating device 401, provided in multiple configurations. In various embodiments, device 401 may be operated by lever 404. Also, FIG. 4 illustrates inlet port 407, through which tissue, fluid, or lipoaspirate may be introduced into device 401.

In various embodiments, device 401 includes an exterior wall surrounding an interior volume, a structure for processing the adipose tissue, and at least one transfer port. In FIG. 4, device 401 is configured such that filtrate exists device 401 through connecting means 405. Filtrate of device 401 can include fluids, waste materials, and adipose tissue particles smaller than the pores of the filter within device 401.

In various embodiments, device 401 may be connected via connecting means 405 to pump 406. Pump 406 may be used to draw filtrate from device 401 and advance it into inlet tube 402. Filtrate traveling through inlet tube 402 may pass through first end 420 and enter device 400. In various embodiments, device 400 may include two chambers separated by a filter (not pictured). The outer chamber may be defined, in part by outer surface 410. The inner chamber may comprise primarily a lumen whose boundaries are defined by a filter (also not pictured).

Device 400 may be provided in a variety of sizes and configurations. In certain embodiments, device 400 may be sized to accommodate a range of filtrate volumes. For example, device 400 may be configured to accommodate filtrate volumes of 0.5, 1, 2, 3, 4, 5, 10, 20, 30, 40, 50, 60, 70, 80, 90, 100, 120, 140, 160, 180, 200, 220, 240, 260, 280, 300, 350, 400, 450, 500, or 1000 ml. These volume values may be used to define a single volume, such as 80 ml, or may be used to define range of volumes, such as from about 100-120 ml. The volume may be configured to accommodate the volume and rate of filtrate input into device 400.

As with device 100, in various embodiments, device 400 may be provided in a variety of materials suitable for tissue processing. For example, device 400 may be made from materials that will enable the device to pass regulatory testing standards, such as ISO 10993-1. Such materials may be sufficiently biocompatible and inert as to not elicit cytotoxic or carcinogenic responses during clinical use. Examples of materials potentially suitable for device 400 may include plastics such as polymers (e.g. polyethylene terephthalate (PET), high density polyethylene (HDPE), polyvinyl chloride (PVC), polypropylene (PP), polyimide (TPI), and acrylonitrile butadiene styrene (ABS)), metals (e.g. stainless steel, titanium alloys, cobalt chromium, nitinol, and copper), or ceramics (e.g. zirconia, or alumina).

The materials of device 400 must be able to withstand stresses of the manufacturing and sterilization processes, and well as stresses endured during clinical use. For example, the materials of device 400 may need to be able to withstand high sterilization temperatures, or pressures generated from suction devices used during the procedure.

Additionally, in certain embodiments, device 400 may comprise one or more materials configured to improve user-experience. For example, the outer surface 410 of device 400 may comprise a transparent material so that users (e.g. surgeons or other medical professionals) may see into the device during use. Furthermore, materials of device 400 may be optimized to minimize cost or to simplify the device manufacturing process.

In various embodiments, filtering performed by device 400 may comprise processing filtrate from device 401. For example, the filter of device 400 may be configured to retain adipose tissue filtered out by device 401 within its inner chamber, while allowing waste materials, such as water and saline, and small undesired components to pass through into its waste collection volume.

In various embodiments, waste materials collected in device 400 may be extracted from device 400 through a variety of means. For example, in various embodiments, pump 466 may generate negative pressure within device 400 and facilitate extraction of waste materials from waste port 464 and into waste collection container 468. In various other embodiments, device 400 may rely on gravity to remove waste material from device 400.

In various embodiments, ports 470 and 464 may be provided as valves that may either be manually operated or one-way valves that open or close in response to certain pressure conditions within device 400. For example, to allow adipose tissue to accumulate, adipose retrieval port 470 may be closed and waste port 464 may be opened to enable the extraction of waste materials, leaving only adipose tissue within the filter of device 400.

In various embodiments, adipose tissue collected with the filter of device 400 may aggregate toward second end 430. In some embodiments, adipose retrieval port 470 may be maintained in a closed position so that adipose tissue may accumulate at second end 430. After tissue processing is complete, waste port 464 may be closed and adipose retrieval port 470 may be opened to establish fluid communication between second end 430 and adipose collection container 480. Then, in some embodiments, device 400 may rely on gravity to transfer adipose tissue into adipose collection container 480. In various embodiments, device 400 may include a negative pressure system, such as a syringe or pump to withdraw adipose tissue from device 400.

In various embodiments, tissue collection container 480 is provided as a syringe. When tissue extraction is desired, negative pressure may be formed within adipose collection container 480 (i.e. by withdrawing the syringe plunger). Alternatively, adipose collection container 480 may be connected to a pump that may generate negative pressure to enable adipose tissue extraction.

In various embodiments, adipose collection chamber 480 may be removed from device 400 and used in clinical applications. For example, adipose collection chamber 480 may be provided as a syringe, onto which a user may connect an injection needle. Afterwards, the user may inject adipose tissue from adipose collection chamber 480 into host sites within a patient, such as into rhytids, lips, scars, or deformities of the face or neck.

Additionally, in certain embodiments, the system of FIG. 2 may be attached to device 401 of FIG. 4 to provide an adipose tissue collection system with multiple adipose collection containers, each harvesting adipose tissue with distinct particle sizes. This system may be desirable in cases where adipose tissue injections sites vary in size. For example, a surgeon may perform buttocks augmentation, cheek enhancement, and facial rhytid correction on the same patient. The surgeon may use adipose tissue collected in device 401 for the buttock enhancement, adipose tissue collected in device 100′ for the cheek enhancement, and adipose tissue collected in device 200 for rhytid correction.

Methods of treatment using the devices described herein are also contemplated and within the scope of the presently claimed inventions.

Other embodiments will be apparent to those skilled in the art from consideration of the specification and practice of this disclosure. It is intended that the specification and examples be considered as exemplary only, with the true scope and spirit of the disclosed devices, systems, and methods being indicated by the following claims.

What is claimed is:

1. A system for processing adipose tissue, comprising:
a tissue container and processing device, including:
an exterior wall surrounding an interior volume;
a structure for processing the adipose tissue; and
at least one transfer port; and
a device for processing filtrate from the tissue container, comprising:
a fluid pathway comprising a first end, a second end, and a fluid lumen extending between the first end and second end for passage of the fluid therebetween;
a first filter disposed within the fluid pathway between the first end and the second end, the filter being configured to collect adipose tissue particles from the filtrate and selectively filter waste material from the fluid;
a waste collection volume surrounding the filter and including a waste port for engagement with a waste removal device; and
a tissue retrieval port in fluid communication with the filter proximate the second end of the fluid pathway.

2. The system of claim 1, wherein the first filter has a pore size of between about 0.2 mm and about 0.05 mm.

3. The system of claim 1, further comprising a second filter positioned downstream of the first filter.

4. The system of claim 3, wherein the second filter has a pore size that is smaller than the pore size of the first filter.

5. The system of claim 1, wherein the first filter is substantially cylindrical.

6. The system of claim 1, wherein the tissue retrieval port is configured to remove collected adipose tissue particles in an open position.

7. The system of claim 1, further comprising a container for the collection of adipose tissue.

8. The system of claim 1, wherein the first filter comprises a mesh.

9. The system of claim 1, further comprising a negative pressure source.

10. The system of claim 9, wherein the negative pressure source is in fluid communication with the fluid pathway.

11. A method of processing adipose tissue, comprising:
selecting a device comprising:
a fluid pathway comprising a first end, a second end, and a fluid lumen extending between the first end and second end for passage of a fluid therebetween;
a first filter disposed within the fluid pathway between the first end and the second end, the filter being configured to collect adipose tissue particles and selectively filter waste material from the fluid;
a waste collection volume surrounding the filter and including a waste port for engagement with a waste removal device; and
a tissue retrieval port in fluid communication with the filter proximate the second end of the fluid pathway;
advancing the fluid through the fluid pathway;
evacuating waste material through the waste port into the waste removal device; and
removing collected adipose tissue particles into a container for collection of adipose tissue.

12. The method of claim 11, wherein advancing the fluid through the fluid pathway comprises applying a suction pressure to the second end of the fluid pathway to draw the fluid into the fluid pathway.

13. The method of claim 11, wherein the first filter has a pore size of between about 0.2 mm and about 0.05 mm.

14. The method of claim 11, wherein the selected device further comprises a second filter positioned downstream of the first filter relative to the first end.

15. The method of claim 14, wherein the second filter has a pore size that is smaller than the pore size of the first filter.

16. The method of claim 11, wherein the tissue retrieval port is configured to remove the collected adipose tissue particles in an open position.

17. The method of claim 11, wherein the container for collection of adipose tissue comprises a syringe.

18. The method of claim 17, further comprising removing the syringe after removing the collected adipose tissue particles.

19. The method of claim 18, further comprising injecting the collected adipose tissue particles into a tissue site.

20. The method of claim 11, wherein the first filter comprises a mesh.
